(19) United States
(12) Patent Application Publication
(10) Pub. No.: US 2010/0145365 A1
(43) Pub. Date: Jun. 10, 2010

(54) FIXTURE FOR MAINTAINING THE TENSION APPLIED TO A SUTURE DURING INTRACORPOREAL PROCEDURES

(76) Inventor: Tyler E. McLawhorn, Winston-Salem, NC (US)

Correspondence Address: BRINKS HOFER GILSON & LIONE/CHICAGO/COOK, PO BOX 10395, CHICAGO, IL, 60610, US

(21) Appl. No.: 12/630,373

(22) Filed: Dec. 3, 2009

Related U.S. Application Data
(60) Provisional application No. 61/120,193, filed on Dec. 5, 2008.

(51) Int. Cl. A61B 17/04

(52) U.S. Cl. 606/144

(57) ABSTRACT

A tension sustaining fixture, a medical system, and related methods for the suturing tissue and the tying of sutures to close a perforation made during an intracorporeal procedure are presented. A tension sustaining fixture generally comprises an elongated tube or sheath; a main body having a first passageway, the main body capable of being coupled to the elongated sheath; a side retaining sleeve coupled to the main body and whose internal surface defines a second passageway intersecting with the first passageway; a closure mechanism engaging the side retaining sleeve; the closure mechanism further defining the second passageway and sized to slidably receive at least one suture; and at least one suture having a proximal and distal end; with at least one end of a suture extending from the first passageway through the second passageway. The closure mechanism being operable between a locked configuration and an unlocked configuration; the locked configuration fixing a suture to maintain the tension applied to the suture.

This application claims the benefit of U.S. Provisional Application Ser. No. 61/120,193 filed on Dec. 5, 2008, entitled “FIXTURE FOR MAINTAINING THE TENSION APPLIED TO A SUTURE DUIRNG INTRACORPOREAL PROCEDURES,” the entire contents of which are incorporated herein by reference.

### FIELD

The present invention relates generally to devices, systems, and methods for sustaining the tension applied to sutures that are being fixed to bodily tissue, such as for closing perforations in the tissue.

### BACKGROUND

Openings or perforations in the walls of internal organs and vessels may be naturally occurring, or formed intentionally or unintentionally. These openings may be used to gain access to adjacent structures of the body, such techniques being commonly referred to as transluminal procedures. For example, culdoscopy was developed over 70 years ago, and involves transvaginally accessing the peritoneal cavity by forming an opening in the cul de sac. This access to the peritoneal cavity allows medical professionals to visually inspect numerous anatomical structures, as well as perform various procedures such as biopsies or other operations, such as tubal ligation. Many transluminal procedures for gaining access to various body cavities using other bodily lumens have also been developed. Natural orifices such as the mouth, nose, ear, anus or vagina may provide access to such bodily lumens and cavities. The bodily lumen(s) of the gastrointestinal tract are often endoscopically explored and can be utilized to provide access to the peritoneal cavity and other body cavities, all in a minimally invasive manner. U.S. patent application Ser. No. 11/946,565 filed Nov. 28, 2007, discloses such a procedure, and is incorporated herein by reference in its entirety.

In order to permanently close naturally occurring, as well as intentionally or unintentionally formed perforations, and allow the tissue to properly heal, numerous medical devices and methods have been developed that employ sutures, adhesives, clips, tissue anchors and the like. Where sutures are used, e.g., with or without anchoring devices, the sutures must be tied or otherwise connected together. Manually tying sutures strands together to close a perforation also can be very complex and time consuming. For example, a significant level of skill and coordination is required by the medical professional, especially when the perforation and sutures are difficult to access within the body, such as in endoscopic or laparoscopic procedures. The numerous difficulties with manually tying sutures are well documented. Likewise, many other endoscopic and laparoscopic procedures require the tensioning and/or tying of sutures and suffer from similar difficulties.

### BRIEF SUMMARY

The present invention provides a tension sustaining fixture, a medical system, and related methods for suturing tissue and for tying sutures to close a perforation made during intracorporeal procedures. One embodiment of a tension sustaining fixture, constructed in accordance with the teachings of the present invention, generally comprises a main body having a first passageway, the main body capable of being coupled along the longitudinal axis of the suturing device. A side retaining sleeve is unitarily formed with or coupled to the main body and has an internal surface defining a second passageway intersecting with the first passageway. A closure mechanism engages the side retaining sleeve. The closure mechanism further defines the second passageway and sized to slidably receive at least one suture having a proximal end and a distal end. The first passageway and the second passageway of the tension sustaining fixture intersect at an angle (α) that preferably has a magnitude less than about 90 degrees, and more preferably less than about 45 degrees.

The distal end of a suture follows a path from the first passageway through the second passageway and the closure mechanism. The closure mechanism is operable between a locked configuration and an unlocked configuration, whereby, the tension applied to the suture can be maintained when the closure mechanism is in its locked configuration.

The amount of friction applied to a suture is negligible when the closure mechanism is in its unlocked configuration. The closure mechanism may be a rotatable member, such as a stopcock, positioned to close the passage way or a pin vise collet having a locking cap. Minute adjustments to the amount of friction applied to the sutures is accomplished by either turning the stopcock partially open or by loosening the locking cap on the pin vise collet, and the tension on the sutures may be manually adjusted.

According to one aspect of the present invention, the intracorporeal procedure may be an endoscopic or laparoscopic procedure using an endoscope and wherein the elongated tube or sheath of the tension sustaining fixture is sized to be inserted through a working channel of the endoscope.

According to another aspect of the present invention, a medical system useful for suturing tissue in a patient during an intracorporeal procedure comprises a suturing device having a handle and an outer sheath that slidably receives the distal end of the sutures for delivery to the tissue within the patient. A tension sustaining fixture having a main body is located between the handle and the outer sheath of the suturing device. A side retaining sleeve intersects with the main body at an angle (α). A closure mechanism is coupled to the side retaining sleeve. At least one suture has a proximal end that is fed from the outer sheath through the tension sustaining fixture to be external to the patient. The closure mechanism is operable between a locked configuration and an unlocked configuration with the amount of tension applied to the sutures being maintained when the closure mechanism is in its locked configuration.

According to yet another aspect of the present invention, a method for closing perforations in the walls of organs and vessels in a patient during an endoscopic or laparoscopic procedure comprises the steps of placing the distal end of an endoscope through an orifice in the patient to a location that is proximate to a bodily vessel; performing the endoscopic or laparoscopic procedure on the vessel; securing the distal end of at least one suture proximate to the perforation made in the wall of the vessel; providing appropriate tension to the suture(s); sustaining the applied tension using a tension sustaining fixture; and securing the sutures to close the perforation.

In this method the tension sustaining fixture has a main body coupled to the suturing device, a side retaining sleeve that intersects with the main body at an angle (α), and a closure mechanism coupled to the side retaining sleeve. The proximal end of at least one suture is fed from the perforation in the bodily vessel through the side retaining sleeve and closure mechanism of the tension sustaining fixture to be external to the patient. The appropriate tension is provided and manipulated manually or through the use of an instrument or robot. The applied tension is sustained by closing the closure mechanism of the tension sustaining fixture. The closure mechanism may be either a rotatable member, such as a stopcock, or a pin vise collet having a locking cap. The tension to the sutures may be adjusted as necessary to effectively close the perforation by opening the closure mechanism and manually adjusting the tension; or by partially opening or closing the closure mechanism when the closure mechanism is a rotatable member.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a perspective view of a system according to one embodiment of the present invention for providing tension to the sutures used to close perforations in the walls of organs and vessels in a patient during an endoscopic procedure;

FIG. 2A is a perspective view of the tension sustaining fixture according to one embodiment of the present invention showing a closure mechanism in its fully open condition;

FIG. 2B is a perspective view of the tension sustaining fixture of FIG. 2A showing the closure mechanism in a partially closed condition;

FIG. 2C is a perspective view of a tension sustaining fixture of FIG. 2A showing the closure mechanism in its fully closed position;

FIG. 3A is a cross-sectional view of the closure mechanism of FIG. 2A in its fully open position taken along line B-B as defined in FIG. 2C;

FIG. 3B is a cross-sectional view of the intersection between the first passage way and the second passage way of the tension sustaining fixture according to one embodiment of the present invention;

FIG. 4A is a perspective view of a tension sustaining fixture according to another embodiment of the present invention;

FIG. 4B is a perspective view of the tension sustaining fixture of FIG. 4A with a locking cap; and

FIG. 5 is a schematic representation of a method for sustaining the tension applied to sutures during an endoscopic procedure.

### DETAILED DESCRIPTION

The following description is merely exemplary in nature and is in no way intended to limit the present invention or its application or uses. It should be understood that throughout the description and drawings, corresponding reference numerals indicate like or corresponding parts and features. In the present application, the term “proximal” refers to a direction that is generally towards a physician during a medical procedure, while the term “distal” refers to a direction that is generally towards a target site within a patient's anatomy during a medical procedure.

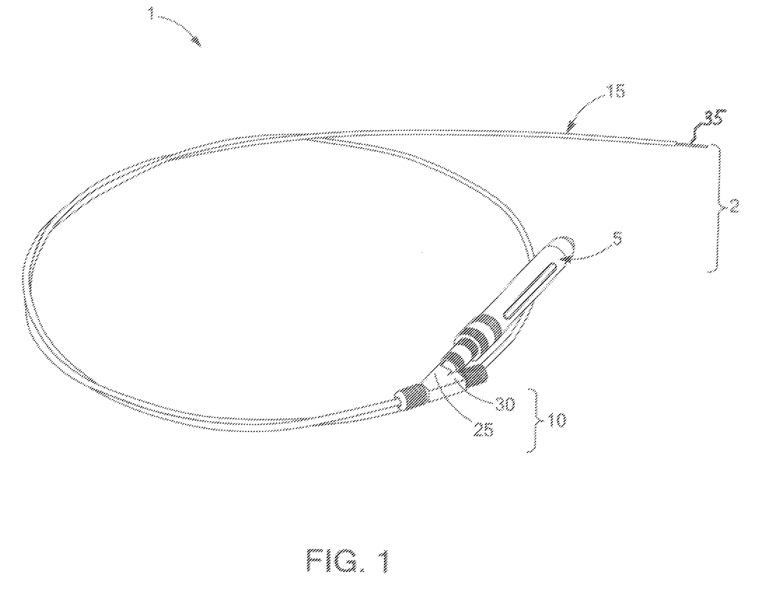

One embodiment of the present invention, constructed in accordance with the teachings herein, generally provides a tension sustaining fixture for use with a suturing device that maintains the tension applied to sutures being fixed to bodily tissue, such as in closing perforations in the tissue, during an intracorporeal procedure. Referring to FIG. 1, the medical system 1 comprises a suturing device 2 and a tension sustaining fixture 10. The suturing device 2 may have a handle 5 and an outer sheath 15. The suturing device may be any type of a device known in the art including those that apply T-anchors, staples, tacks, clips or the like. According to one aspect of the present invention, the intracorporeal procedure may be an endoscopic or laparoscopic procedure using an endoscope where the suturing device 2 and the tension sustaining fixture 10 are integrated together such that the elongated tube or sheath 15 is shared by and is considered to be part of both the suturing device 2 and the tension sustaining fixture 10. The elongated tube or sheath 15 is sized to be inserted through a working channel of the endoscope.

One example of a suturing device 2 is one that utilizes a set of tissue anchors having an anchoring member attached to one or more sutures 35. Such exemplary devices are disclosed in U.S. patent application Ser. No. 11/946,565 filed on Nov. 28, 2007, and exemplary staples are disclosed U.S. patent application Ser. No. 12/191,277 filed on Aug. 13, 2008, and exemplary tacks are disclosed in U.S. patent application Ser. No. 12/428,226 filed on Apr. 22, 2009, the contents of each are hereby incorporated by reference in their entirety. These tissue anchors are loaded into a delivery needle, and here the suture(s) are fed through the outer sheath 15 of the suturing device 2. The crossbar/anchoring member of the tissue anchor is passed through the bodily wall adjacent to the periphery of the perforation in the wall. Thus each tissue anchor ends up being on the distal side of the bodily wall with the opposite ends of the suture 35 remaining on the proximal side of the bodily wall. The sutures 35 are then tensioned to reduce the distance between the tissue anchors and to compress the bodily wall proximate to the perforation. Finally, the ends of the sutures 35 are fixed together in order to maintain the compression of the bodily wall and to close the perforation.

The ends of the sutures 35 may be manually tied or a suture lock may be employed (see, e.g., U.S. patent application Ser. No. 12/125,525 filed on May 22, 2008 and U.S. patent application Ser. No. 12/191,001 filed on Aug. 13, 2008, the contents of which are hereby incorporated by reference in their entirety). Although this example describes the use of T-anchors, one skilled in the art will recognize that other types of suturing devices and methods may be utilized without departing from the scope of the present invention, including staples (see, e.g., U.S. patent application Ser. No. 12/191,277), tacks (see, e.g., U.S. patent application Ser. No. 12/428,226), clips, and similar devices.

Sutures 35 are generally placed in tension by the natural elasticity of the bodily tissue pulling the sutures 35 distally. At the same time the sutures 35 may be pulled in the proximal direction to further apply and adjust suture tension, lock suture position, orient the suturing device, and to further close the perforation. This tension may be applied and manipulated by hand, using a device or instrument, or by a robot.

The tension sustaining fixture 10 is located at the proximal end of the system 1, and preferably between the handle 5 and the outer sheath 15 of the suturing device 2. The tension sustaining fixture 10 may be unitarily formed with, integrally mounted on, or fastened to the suturing device 2.

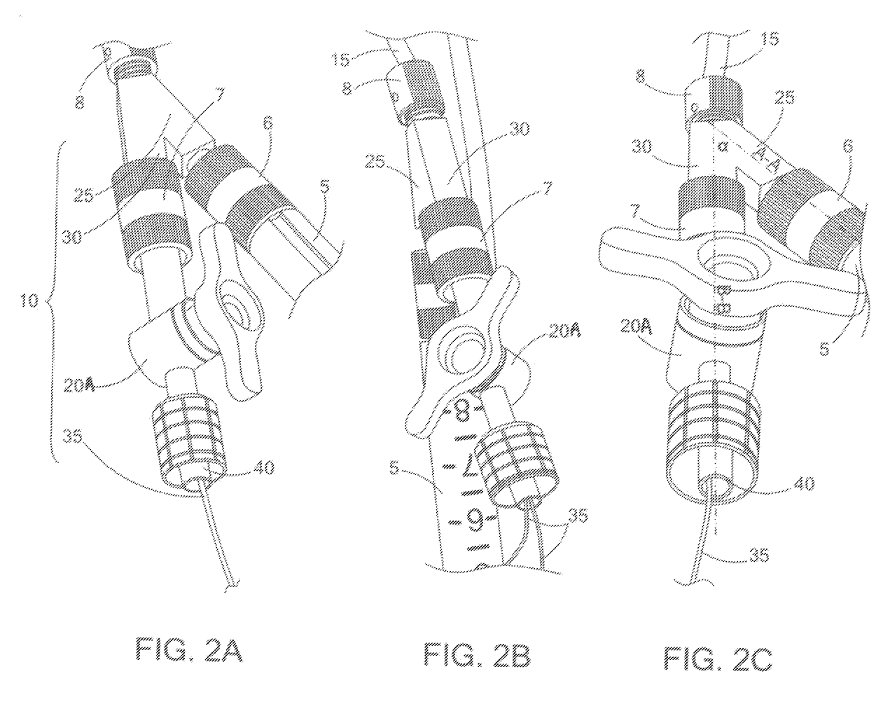

Referring now to FIGS. 2A-2C, 3A, 3B, and 4A, the tension sustaining fixture 10 comprises a main body 25 having a first passageway 31, the main body 25 capable of being in communication with the elongated lumen or sheath along the longitudinal axis A-A of the suturing device 5. A side retaining sleeve 30 is integrally formed with or coupled to the main body 25 and has an internal surface that defines a second passageway 32 intersecting with the first passageway 31. A closure mechanism 20 engages the side retaining sleeve 30. The closure mechanism 20 defines a passageway 40 extending as part of the second passageway 32 and is sized to slidably receive at least one suture 35. The suture 35 used in the suturing device 2 has a proximal and distal end. The distal end of a suture 35 extends through a catheter or needle 11 near the area in the lumen to be sutured. The proximal end of a suture 35 is extended from the first passageway 31 through the second passageway 32/40 of the tension sustaining fixture 10.

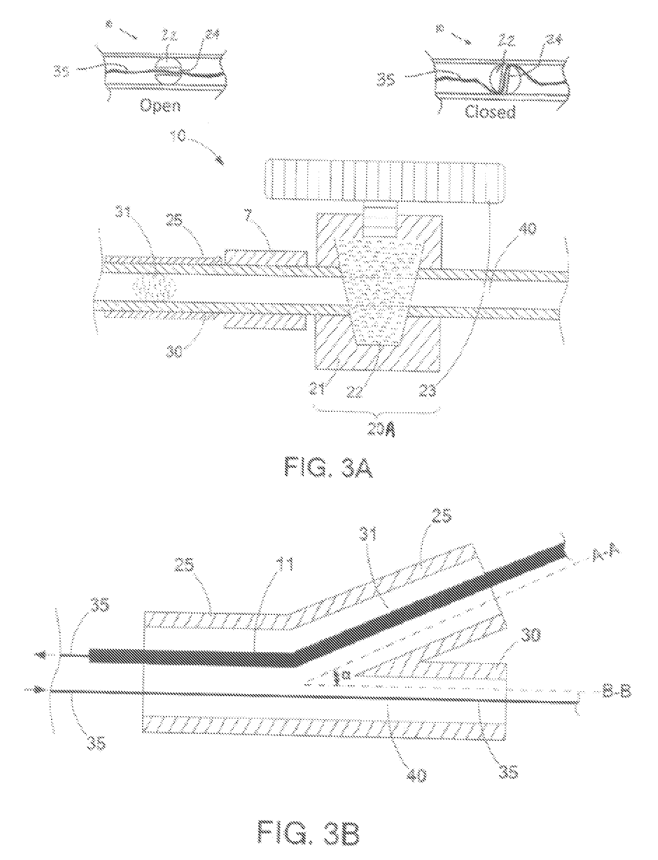

As also shown in FIGS. 2A-2C and FIG. 3A, the main body 25 of the tension sustaining fixture 10 and the handle 5 of the suturing device 2 are in-line with each other along axis A-A. The side retaining sleeve 30 and closure mechanism 20 of the tension sustaining fixture 10 are in-line with each other along axis B-B. The first passageway of the main body 25 positioned along axis A-A and the second passageway 40 of the side retaining sleeve 30 are positioned along axis B-B and intersect with each other making an angle (α) as shown in FIGS. 2C and 3B. This angle (α) is less than about 90 degrees, with less than about 45 degrees being preferred.

The main body 25 of the tension sustaining fixture 10 may be integrally formed with or coupled to both the handle 5 and outer sleeve 15 of the suturing device 2. The retaining sleeve 30 and the main body 25 are preferably unitarily formed. The retaining sleeve 30 may be unitarily formed with or integrally coupled to the closure mechanism 20. The coupling of the main body 25 of the tension sustaining fixture 10 to the handle 5 and the outer sleeve 15 of the suturing device 2 and the coupling of the retaining sleeve 30 to the closure mechanism 20 of the tension sustaining fixture 10 can be accomplished using any type of fastener (see 6, 7, & 8, respectively) known to one skilled in the art, such as threaded connectors, nuts, or quick connect fittings, among others.

The closure mechanism is operable between a locked configuration (FIG. 2C) and an unlocked configuration (FIG. 2A). The amount of tension applied to the sutures 35 is maintained when the closure mechanism is in a locked configuration (FIG. 2C) or a partially locked configuration (i.e. between the unlocked and fully locked configurations). The amount of friction placed on the sutures 35 by the closure mechanism 20 is negligible when the closure mechanism is in an unlocked configuration. Once tension is applied to the suture 35 and is desired to be maintained, the closure mechanism 20 can be closed in order for the tension to be maintained.

In one embodiment of the present invention, the closure mechanism 20 may be a rotatable member 20A positioned to close the passage way, such as a stopcock 20A as shown in FIGS. 2A-2C. A stopcock 20A may be comprised of a rotatable key 22, a fixed body 21, and a handle 23. The handle 23 is coupled to the key 22 is used to rotate the key 22 within the confines of the body 21 from its fully open position to its fully closed position (i.e. unlocked and locked configurations). It is also possible to partially rotate the key 22 to a position between the fully open and closed positions. The suture 35 is fed through the opening (indicated by dotted lines) in the key 22 of the stopcock. When the rotatable member or stopcock 20A is closed, the suture becomes “pinched” or otherwise frictionally engaged between the body 21 and the key 22, thereby maintaining the tension on the suture. Opening the stopcock 20A either partially or fully allows the suture to slide through the opening in the key 22 and hence through the second passageway 32/40, and allows the user to manually adjust the tension on the sutures until the appropriate tissue approximation is obtained.

The applied tension can then be maintained by closing the stopcock 20A as shown in FIG. 2C. Once the stopcock 20A is closed, the tension is sustained, and the user's hands are free to operate the suturing device 2 in order to provide a permanent lock on the suture. The tension on the sutures can be released by simply opening the stopcock 20A as shown in FIG. 2A. In the open position, adjustments can be made to the applied tension, which then can be sustained by closing the stopcock 20A. In this embodiment, minute adjustments in the amount of friction applied to the sutures 35 can be made by slightly turning the stopcock 20A from its closed configuration in the direction of its open configuration as shown in FIG. 2B. The user may provide a slight pulling action on the sutures at this time to achieve the desired level of tension and tissue approximation. The stopcock 20A is also capable of engaging the suture 35 such that tension on the suture 35 may be increased and then automatically maintained.

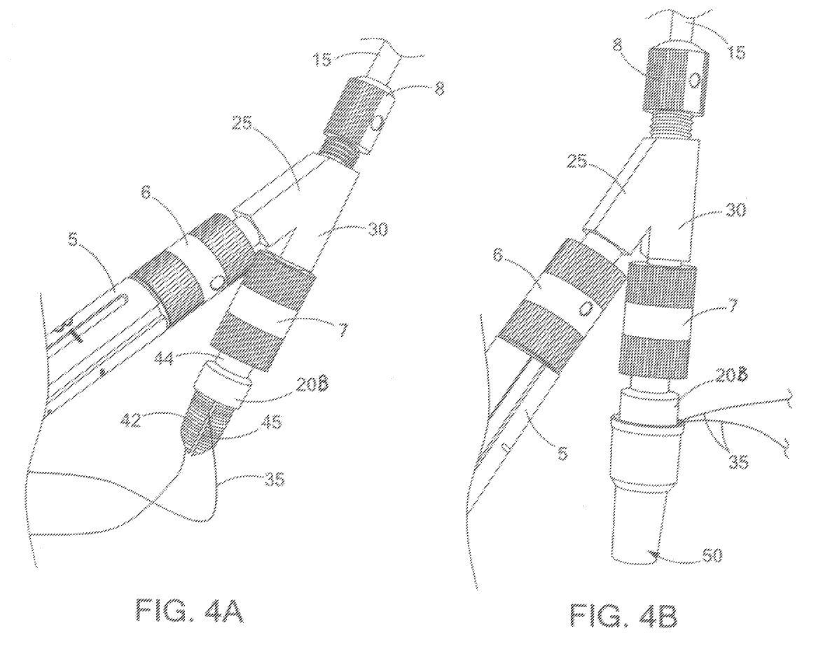

In another embodiment of the present invention, the closure mechanism 20 may be a pin vise collet 20B as shown in FIG. 4A. A pin vise collet 20B is a type holding device, similar to a chuck, that includes a slotted cylindrical member or collar 42 having slots 45 that are adjustable in size via a locking cap 50 to exerts a strong clamping force on the suture 35. The collet 20B may have one or more slits 45 in its collar 42. The sutures 35 are fed through the tubular body 44 of the pin vise collet 20B, and then are pulled to one side through one or more of the slits 45 made in the collar 42 of the collet 20B. The appropriate tension to achieve tissue approximation may then be manually obtained by the operator pulling on the sutures 35. Once the desired level of tension is obtained, the tension may be sustained by placing the locking cap 50 onto the collet 20B as shown in FIG. 4B. The inner diameter of the locking cap 50 is threaded and sized relative to the outer threads of the collar 42 in order to pinch or otherwise frictionally engage the suture 35. Minute changes in the level of friction can be made by slightly loosening the locking cap 50, and the tension may be adjusted by pulling or releasing the sutures 35 to obtain the desired level of tension and tissue approximation. Retightening the locking cap 50 will then sustain the new level of tension applied to the sutures 35.

The main body 25, side retaining sleeve 30, and closure mechanism 20 of the tension sustaining fixture 10 may be comprised of any known material approved for medical applications. This material may include, but not be limited to stainless steels, titanium, nitinol, ceramic materials, and plastic materials. Examples of plastic materials suitable for this application include polycarbonates, polyamides, polytetrafluorethylenes, polyethylene ether ketones, polyvinylchlorides, polyimides, polyurethanes, and polyethylenes, among others. The sutures 35 may be comprised of any material known to one skilled in the art. Materials, such as 2-0 silik, 2-0 Ti-Cron, 4-0 polypropylene, 5-0 polypropylene, 6-0 polypropylene, and 7-0 polypropylene are preferred.

It is another objective of the present invention to provide a medical system useful for suturing tissue in a patient during an intracorporeal procedure. This medical system basically comprises the combination of the suturing device 2 and the tension sustaining fixture 10, such as those described above. As noted above, the tension sustaining fixture 10 and the suturing device 2 may be integrally and/or unitarily formed as a single unit, or an existing suturing device 2 may be modified to include the tension sustaining fixture 10.

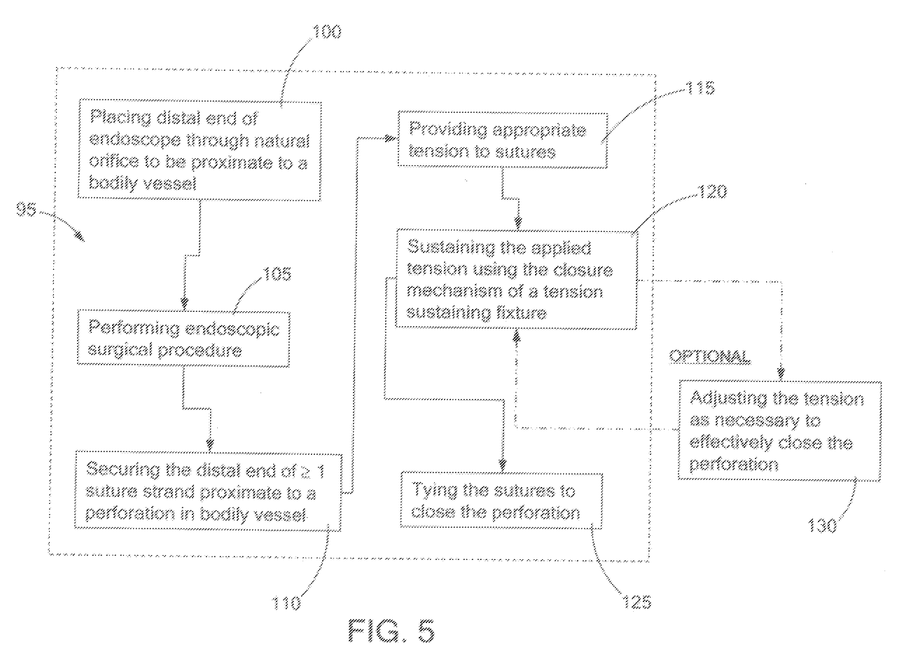

It is another objective of the present invention to provide a method for closing perforations in the walls of organs and vessels in a patient during an endoscopic or laparoscopic procedure. Referring now to FIG. 5, this method 95 comprises the steps of placing 100 the distal end of an endoscope through an orifice in the patient to a location that is proximate to a wall of a bodily lumen; performing 105 the endoscopic or laparoscopic procedure on the wall of the lumen; securing 110 the distal end of at least one suture proximate to the perforation made in the wall of the lumen; providing 115 appropriate tension to the sutures; sustaining 120 the applied tension using a tension sustaining fixture; and tying 125 the sutures to close the perforation.

The proximal end of at least one suture is fed from the perforation in the bodily vessel through the side retaining sleeve and closure mechanism of the tension sustaining fixture to be external to the patient. The appropriate tension is then provided and manipulated either manually or through the use of an instrument or a robot.

The applied tension may be sustained by closing the closure mechanism 20 of the tension sustaining fixture 10. Such a closing mechanism may be a rotatable member 20A, such as a stopcock 20A, or a pin vise collet 20B with a locking cap. The method further comprises the step of adjusting 130 the tension as necessary to effectively close the perforation. This type of adjustment may be accomplished by partially opening the closure mechanism.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

It will be recognized by those skilled in the art that, while the methods described in this disclosure generally include placing the tissue devices in tissue through an internal bodily lumen, it will be recognized that these systems, devices and methods may be used on any layer of material (e.g. fabrics, cloth, polymers, elastomers, plastics and rubber) that may or may not be associated with a human or animal body and a bodily lumen. For example, the systems, devices and methods can find use in laboratory and industrial settings for placing devices through one or more layers of material that may or may not find application to the human or animal body, and likewise closing holes or perforations in layers of material that are not bodily tissue.

1. A tension sustaining fixture for use with a suturing device during an intracorporeal procedure, the tension sustaining fixture having:
an elongated tube or sheath;
a main body having a first passageway, the main body capable of being coupled to the elongated sheath;
a side retaining sleeve coupled to the main body and whose internal surface defines a second passageway intersecting with the first passageway;
a closure mechanism engaging the side retaining sleeve; the closure mechanism further defining the second passageway and sized to slidably receive at least one suture; and
at least one suture having a proximal and distal end; with at least one end of a suture extending from the first passageway through the second passageway; and
the closure mechanism being operable between a locked configuration and an unlocked configuration; the locked configuration fixing a suture to maintain the tension applied to the suture.

2. The tension sustaining fixture of claim 1, wherein the first passageway and the second passageway intersect at an angle (α) having a magnitude less than about 90 degrees.

3. The tension sustaining fixture of claim 1, wherein the amount of tension placed on a suture by the closure mechanism is negligible when the closure mechanism is in an unlocked configuration.

4. The tension sustaining fixture of claim 1, wherein the closure mechanism is selected as one from the group of a rotatable member positioned to close the passage way and a pin vise collet having a cap in a tightened or locked position.

5. The tension sustaining fixture of claim 4, wherein the rotatable member is a stopcock.

6. The tension sustaining fixture of claim 5, wherein the stopcock comprises a rotatable key, a fixed body, and a handle.

7. The tension sustaining fixture of claim 5, wherein minute adjustments to the amount of tension applied to a suture is accomplished by rotating the stopcock to a partially open or closed position.

8. The tension sustaining fixture of claim 4, wherein the pin vise collet comprises at least one slit in its body or collar that engages a tensioned suture when the cap is tightened.

9. The tension sustaining fixture of claim 8, wherein minute adjustments to the amount of tension applied to a suture is accomplished by loosening the locking cap and manually adjusting the tension applied.

10. The tension sustaining fixture of claim 1, wherein the intracorporeal procedure is an endoscopic procedure using an endoscope and wherein the elongated tube or sheath of the tension sustaining fixture is sized to be inserted through a working channel of the endoscope.

11. A medical system for suturing tissue in an intracorporeal procedure in a patient, the medical system comprising:
a suturing device having a handle and an outer sheath that slidably receives the distal end of the sutures for delivery to the tissue within a patient;
a tension sustaining fixture having a main body located between the handle and the outer sheath of the suturing device, a side retaining sleeve intersecting with the main body at an angle (α); and a closure mechanism coupled to the side retaining sleeve; and
at least one suture whose proximal end follows a path from the outer sheath through the tension sustaining fixture to be external to the patient;
wherein the closure mechanism is operable between a locked configuration and an unlocked configuration;
wherein the amount of tension applied to the sutures is maintained by fixing the position of the suture when the closure mechanism is in a locked configuration.

12. The system of claim 11, wherein the magnitude of angle (α) is less than about 90 degrees.

13. The system of claim 11, wherein the amount of tension placed on the sutures by the closure mechanism is negligible when the closure mechanism is in an unlocked configuration.

14. The system of claim 11, wherein the closure mechanism is selected as one from the group of a rotatable member positioned to close the passage way and a pin vise collet having a cap in a tightened or locked position.

15. The system of claim 14, wherein minute adjustments to the amount of tension applied to the sutures is accomplished by partially opening or closing the rotatable member.

16. The system of claim 14, wherein minute adjustments to the amount of tension applied to a suture is accomplished by loosening the locking cap on the pin vise collet and manually adjusting the tension applied.

17. A method for closing perforations in the walls of organs and vessels in a patient during an endoscopic or laparoscopic procedure, the method comprising the steps of:
placing the distal end of a suturing device through an orifice in the patient to a location that is proximate to a bodily vessel;
performing the endoscopic or laparoscopic procedure on the vessel;
securing the distal end of at least one suture proximate to the perforation made in the wall of the vessel;
providing appropriate tension to the sutures;
sustaining the applied tension using a tension sustaining fixture; and
securing the sutures to close the perforation;
wherein the tension sustaining fixture has a main body coupled to the suturing device, a side retaining sleeve that intersects with the main body, and a closure mechanism coupled to the side retaining sleeve;
wherein the proximal end of at least one suture is fed from the perforation in the bodily vessel through the side retaining sleeve and closure mechanism of the tension sustaining fixture to be external to the patient.

18. The method of claim 17, wherein the appropriate tension is provided through the use of one selected from the group of manual, instrument, or robotic manipulation.

19. The method of claim 17, wherein the applied tension is sustained by closing a closure mechanism selected as one from the group of a rotatable member positioned to close the passage way and a pin vise collet having a cap in a tightened or locked position.

20. The method of claim 18, wherein the method further comprises the step of adjusting the tension as necessary to effectively close the perforation.
